SEQUENCE LISTING (3 sequences)

SEQ ID NO: 1 - DNA, 25 bp, HCMV AD169
ggatccgcatggcattcacgtatgt

SEQ ID NO: 2 - DNA, 25 bp, HCMV AD169
gaattcagtggataacctgcggcga

SEQ ID NO: 3 - DNA, 21 bp, HCMV AD169
gccaccgcagatagtaagcgc

(19) United States
(12) Patent Application Publication
(10) Pub. No.: US 2005/0032049 A1
(43) Pub. Date: Feb. 10, 2005

(54) Electrochemical method for detecting nucleic acids

(76) Inventors: Fatima Azek, Saint Maurice (FR); Pierre Brossier, Quetigny (FR); Martine Joannes, Carrieres Sur Seine (FR); Benoit Limoges, Bretigny Sur Orge (FR)

Correspondence Address: Blakely Sokoloff Taylor & Zafman, Seventh Floor, 12400 Wilshire Boulevard, Los Angeles, CA, 90025, US

(21) Appl. No.: 10/204,533

(22) PCT Filed: Feb. 20, 2001
(86) PCT No.: PCT/FR01/00488

(30) Foreign Application Priority Data
Feb. 24, 2000 (FR) 00/02,323

(51) Int. Cl. C12Q 1/68

(52) U.S. Cl. 435/6; 205/777.5

(57) ABSTRACT

The invention concerns a novel method for detecting and/or quantifying nucleic acid molecules in a biological sample by electrochemistry and a kit of reagents for implementing said method.

The present invention relates to a novel method for detecting and/or assaying nucleic acid molecules in a biological sample by electrochemistry, and also to a kit of reagents for carrying out this method. A particular embodiment of this method makes it possible to detect the presence of contamination with a pathogenic agent in a biological sample.

The presence of pathogenic agents in an organism can, today, be detected by several methods. Most of these use a prior step of amplification, in general by polymerase chain reaction (PCR), which makes it possible to specifically amplify DNA fragments of viral genome. This extremely sensitive method makes it possible to detect a very small number of molecules in the organism and, under certain conditions, to quantify the number of copies of the genome of the agent which are initially present.

This technique is, a priori, easy to use and makes it possible to obtain reliable results.

The methods conventionally used to analyze PCR products are electrophoresis, with staining of the DNA with ethidium bromide (ETB), or hybridization tests using probes labeled, for example, with radioactive or luminescent compounds or compounds detectable by colorimetry. These hybridization techniques are widely used for medical diagnoses.

Recently, other methods have been developed for obtaining PCR products which can be directly analyzed. For example, specifically labeled primers can be used, the signal emitted by the amplified fragments subsequently being analyzed using relatively laborious systems. In particular, the primers used for the amplification can carry a fluorophore, the measurement of fluorescent emission from which will make it possible to determine the amount of DNA amplified. Moreover, a limitation of these methods remains the difficulty in carrying them out easily, insofar as they require the use of large pieces of equipment. Moreover, the risk of interference also limits these methods.

Since DNA possesses electroactive nucleic bases, electrochemical detection systems have also been developed, which take advantage of this property to directly detect the hybridized DNA, without having to involve a label. In general, the DNA is immobilized on an electrode, and the difference in electric current measured before and after hybridization is related to the amount of DNA attached to the electrode. The use of such a method is described in patent application WO 93/20230. However, this direct detection without label is not very sensitive.

In order to further improve sensitivity, other approaches have been developed, making use of an electroactive probe molecule or an electroactive label. Thus, Palanti et al. (1996, Analytical Letters, 29, pp. 2309-31) describe various electroactive compounds which can associate with DNA and thus be detected by oxidation or reduction by applying a potential to the electrode. Transition metal complexes, antibiotics, acridine or benzamide dyes and other DNA-intercalating agents have thus been used. These electroactive probes or labels have better redox properties than DNA. Their use makes it possible to obtain a higher signal/noise ratio and better sensitivity. The detection threshold for the DNA of a human immuno deficiency virus type 1, obtained with such methods, was of the order of a nanomole (Wang et al., 1996, Analytical Chemistry, 68, 2629-34).

The present invention makes it possible to improve the sensitivity of an electrochemical detection of DNA, through the use of an enzymatic label capable of rapidly transforming an inactive substrate into an electrochemically detectable compound, at the surface of the electrode.

Thus, a subject of the present invention is a method for detecting and/or assaying nucleic acids in a sample, directly or after amplification of a specific nucleic acid, in particular specific for pathogenic agents, by electrochemistry, comprising the following steps:
a. a nucleic acid is attached to electrodes,
b. a nucleic acid complementary to the attached nucleic acid is specifically hybridized, said complementary nucleic acid containing a recognition agent,
c. the agent complementary to the recognition agent of (b) is added, said complementary agent being coupled to an enzyme,
d. a substrate for said enzyme is added such that the action of said enzyme on said substrate leads to the formation of an electroactive compound which can be detected by measuring the variation in faradic current after application of a potential to the electrode.

A step for detecting the electroactive compound may also be added to this method:
e. the accumulation of the electroactive compound thus generated is detected by measuring the variation in faradic current after application of a potential to the electrode.

In step d, it is possible to have a cascade of enzymatic reactions before formation of the electroactive compound. If different enzymes are coupled to the complementary agent defined in step c., the compound obtained after action of the first enzyme on the substrate added may, itself, be a substrate for another enzyme, and so on, until the electroactive compound is finally contained.

The current can be measured using electrochemical techniques such as linear, cyclic, normal pulse, differential pulse or square wave voltammetry, or alternatively amperometry, chronoamperometry, coulometry, chronocoulometry, or anodic stripping or cathodic stripping potentiometry.

The nucleic acid attached to the electrode may be the nucleic acid the detection of which is sought (target) or may be a probe. In this case, the target nucleic acid is added subsequently. It is labeled with the recognition agent. It is possible to carry out such a labeling, for example during an amplification in particular by PCR using labeled primers.

As specified above, the target nucleic acid may have been amplified, in particular by PCR. The nucleic acid which is adsorbed onto the surface of the electrode is preferably in single-stranded form, whether it is naturally so or it is a denatured double-stranded nucleic acid, in order to allow hybridization of the complementary nucleic acid. Such a denatured double-stranded nucleic acid is also considered to be single-stranded for the purpose of the invention. If the target nucleic acid is double-stranded, the hybridization is understood to be the formation of a triple helix nucleic acid complex.

For the purpose of the invention, a “probe” is defined as being a single-stranded nucleic acid fragment or a denatured double-stranded fragment comprising, for example, from 12 bases to a few kilobases, in particular from 15 to a few hundred bases, preferably from 15 to 5-0 or 100 bases, which has a specificity of hybridization under given conditions so as to form a hybridization complex with a target nucleic acid.

The term “nucleic acid” is in particular intended to mean DNA, RNA or PNAs. This nucleic acid may be in single-stranded form or in double-stranded form. It may also be modified, particularly at the level of the bonds between the various elements. In particular, phosphorothioate bonds rather than phosphodiester bonds may be envisioned. It may also be labeled, radioactively or with fluorescent or luminescent compounds or with organometallics.

The term “recognition agent” is intended to mean a compound which can be coupled to nucleic acids and can be recognized specifically by another compound, which is called a complementary agent. Examples of recognition agents and complementary agents which may be used comprise in particular antigen/antibody, hapten/antibody or biotin/streptavidin or avidin complexes. The latter agents will be preferred for carrying out the method according to the invention.

The term “biological sample” is intended to mean any sample containing biological material. This in particular comprises cell cultures maintained in vitro, or samples which may be obtained from an animal or from a human (biopsies, blood samples).

The applicant has demonstrated that the method according to the invention makes it possible to obtain a very low sensitivity threshold for detecting nucleic acids, in particular of amplified DNA, of the order of an attomole. Specifically, this method has the advantage, compared to the methods described above, of having several amplification steps:
the step of amplification of the DNA by PCR, when this is carried out,
an “enzymatic amplification” step, when the substrate for peroxidase is added. In fact, the variation in faradic current measured will be due to the concentration of an electroactive compound present in the solution, said concentration possibly being modified by adjusting the substrate/enzyme incubation time,
the optional enzymatic cascade step as described above.

Moreover, a molecule of complementary agent may be coupled to several molecules of enzymes, which is a further source of signal amplification.

The detection of the nucleic acid by the method according to the invention is in fact carried out by detecting an electrochemical compound rather than by detecting the nucleic acid, and is based on the amplification of a signal rather than on the amplification of the target.

Moreover, the method according to the invention can be readily miniaturized and/or automated, which reduces the risks of contamination of the samples, and makes it possible to carry out analyses at reduced cost. It is even advantageous to perform such a miniaturization.

The Applicant has in fact shown that, surprisingly, the sensitivity of the system is improved when working in small volumes. The term “small volumes” is intended to mean volumes of between a few microliters and a few tens of microliters, in particular from 5 to 50 μl, preferably of 10 μl.

In fact, insofar as the variation in faradic current at the surface of the electrode is detected, it is advantageous to increase the S/V (electrode surface/solution volume) ratio in order to obtain a better signal.

The electrodes will preferably be electrodes which have been screen printed with a carbon-based ink, and which may or may not be modified. Such electrodes have previously been described in the state of the art, for example in patent application WO 93/20230 or in Bagel et al., (1997, Analytical Chemistry, 69, pp. 4688-94). In particular, use is made of screen printed electrodes, with an ink which contains carbon and styrene derivatives. A preferred derivative is polystyrene. The graphite/polystyrene ratio (by weight) is between 1/10 and 10/1, preferably between 1/5 and 5/1, even more preferably between 1/2 and 2/1. A ratio of between 5/4 and 7/4, in particular 3/2, is most particularly preferred. The solvent used must allow good homogenization of the compounds present in the ink and must be able to “dry” rapidly (approximately 30 minutes to 3 hours), in particular by evaporation. The evaporation preferably takes place at ambient temperature. The screen printing is preferably carried out on flexible sheets of polyester or of PVC. These descriptions correspond to particular examples of electrodes, but it is understood that those skilled in the art will be able to optimize them depending on the desired use.

The invention is in particular characterized in that the nucleic acids present in the solution which is analyzed (which may be a biological sample) are adsorbed specifically onto the electrode.

To do this, an attaching buffer, characterized in that it contains 1.5 M of ammonium acetate, is used. Said buffer may in particular be based on PBS (4.3 mM NaH₂PO₄; 15.1 mM Na₂HPO₄; 50 mM NaCl, pH 7.4) or on Tris (50 mM Tris; 1 mM MgCl₂.6H₂O; 50 mM NaCl, pH 7.4).

When the probe labeled with the recognition agent is added, it is preferable for it to attach only to the target DNA and for it not to attach non-specifically to the electrodes. In fact, such an attachment would lead to the subsequent binding of the complementary agent and to the formation of the electroactive compound, after addition of the substrate for the enzyme. A falsely positive reaction would then be obtained.

The hybridization buffer must therefore allow specific hybridization of the labeled probe to the target DNA. Conventional hybridization buffers, as described in Sambrook et al. (Molecular cloning: a laboratory manual, 1989, 2^{nd} Edition, Cold Spring Harbor Laboratory Press, Cold Spring Harbor, N.Y., USA, see in particular p. 9.54), are used. A hybridization buffer which can be used in the method according to the invention contains 6×SSC, 0.1% SDS.

Preferably, if a single enzyme is used, it has oxidase activity. It may, for example, be a peroxidase or a glucose oxidase, but it may also be another type of enzyme, such as a hydrolase, for instance alkaline phosphatase. A peroxidase is preferably used, in particular horseradish peroxidase (HRP). A preferred substrate for peroxidase linked to streptavidin will be ortho-phenylenediamine (OPD). The peroxidase catalyzes interaction of the OPD with H₂O₂ to give a colored, electroactive, water-soluble compound: 2,2′-diaminoazobenzene (DAA). Other substrates for peroxidase may, however, be used, for example tetramethylbenzidine (TMB), derivatives of o-phenylenediamine and diaminobenzenes, hydroquinone and derivatives thereof, 2,2′-azinobis(3-ethylbenzothiazoline-6-sulfonic)acid, phenoxazines and the like, the 4-aminoantipyrine/phenol or 4-aminoantiopyrine/aniline systems, ferrocenes and the like.

When the detection is carried out after an enzymatic cascade, it is important for the final enzyme used to transform the final substrate into an electroactive compound. The preceding enzymes, which serve to amplify the signal, may be conventional enzymes used in biology and active under the experimental conditions. Advantageous enzymes are, for example, enzymes which allow hydrolysis of sugars, such as glucosidases and related enzymes.

The invention also relates to a kit of reagents, for carrying out the method according to the invention, which contains:
a. an attaching buffer for attaching the nucleic acid to the electrodes,
b. a hybridization buffer for the specific hybridization of the complementary nucleic acid to the nucleic acid attached to the electrodes,
c. a recognition agent in order to label the complementary nucleic acid,
d. an agent complementary to the recognition agent (d), coupled to an enzyme, and
e. a substrate for said enzyme, leading to the formation of an electroactive compound which allows the variation in faradic current of the solution to be measured.

Other elements may be added, in particular primers for amplifying the target nucleic acid, or a buffer for denaturing the double-stranded nucleic acid when the starting nucleic acid is double stranded and a single-stranded nucleic acid is attached to the electrodes. The method according to the invention may be used for detecting and/or assaying DNA originating from varied sources. In particular, mention may be made of the detection of DNA of bacterial, viral or cellular origin.

The method according to the invention may, in fact, be used for detecting the overexpression or the underexpression of certain genes involved in cancerous phenomena, for example after reverse transcription steps—amplification from messenger RNA which can be prepared from a biopsy. In fact, the method according to the invention makes it possible to quantify the target nucleic acid, on condition of there being an internal standard.

The method according to the invention may also be used for detecting and/or quantifying possible bacterial contaminations, should this be in food samples (in particular contaminations with Salmonella, Listeria, enterohemorragic E. coli 0157 and/or 011 etc.). The method according to the invention is also of great use for detecting and diagnosing bacterial infections in humans or in veterinary medicine. Mention may be made of M. tuberculosis infections which may be detected using human sputum, or the characterization of other infections for which a rapid and reliable result is desired.

The method according to the invention is of particular use for detecting viruses in the organism, making it possible to obtain excellent sensitivity and therefore to detect a very small number of copies of viral DNA. In particular, it is possible to detect the presence of a virus in a biological sample by following a protocol comprising the following steps:
a. specific amplification of the DNA of the biological sample, preferably by PCR using primers specific for the virus being sought,
b. isolation and analysis of the DNA obtained after amplification using a method according to the invention or using a kit according to the invention.

The method according to the invention also makes it possible to detect, at the same time, the presence, in a sample, of nucleic acids originating from varied sources or organisms. In fact, the principle of the method according to the invention is the detection of a faradic current specific for the electrochemical compound formed by the action of the enzyme on the substrate.

When the intention is to detect the presence, in a sample, of nucleic acid originating from various sources, the following protocol may be followed:
a. optional amplification and attachment of the target nucleic acids to the electrodes,
b. hybridization with the nucleic acids complementary to the targets, each one of them being linked to a different recognition agent,
c. addition of the agents complementary to the recognition agents, coupled to different labels,
d. addition of the various substrates for the enzymes in order to generate the various electroactive compounds,
e. measurement of the faradic current corresponding to the various electroactive compounds, at the surface of the electrode, by applying the potential specific for each compound.

The various labels of step c are enzymatic or other labels. The substrates for the enzymes generate different electroactive compounds, the other labels being specific redox labels. The target nucleic acids attached to the electrodes are preferably single-stranded.

Since the generation of the faradic current is linked to the presence of the specific electroactive compound, it is therefore possible to deduce therefrom the presence or absence of the nucleic acids in the starting sample.

It is understood that the method example given above may be modified, for example by attaching the probes specific to the various nucleic acids to the electrodes, and by coupling the target nucleic acids to the various recognition agents, for example in a PCR amplification step.

This method therefore makes it possible to rapidly and easily identify microbial or viral contaminants in food samples, or in a biological sample.

To carry out the method according to the invention during the analysis of a biological sample, it is generally advised to perform a prior specific amplification of the DNA the detection of which is being sought. The PCR reaction may be performed directly on the sample, or after prior purification of the DNA of the sample. One or other technique is chosen, depending on the amount of sample available and on the aims being sought by the individual carrying out the method according to the invention. Those skilled in the art are aware of the techniques to be used to isolate the DNA from a biological sample.

When the method is used in an automated and/or miniaturized system, the isolation of the DNA may be carried out directly on the electrodes, for example using the teaching of patent WO 97/41219, the DNA thus isolated then possibly being subsequently amplified by PCR.

### DESCRIPTION OF THE FIGURES

FIG. 1: Diagrammatic representation of the detection method according to the invention on a screen printed electrode.

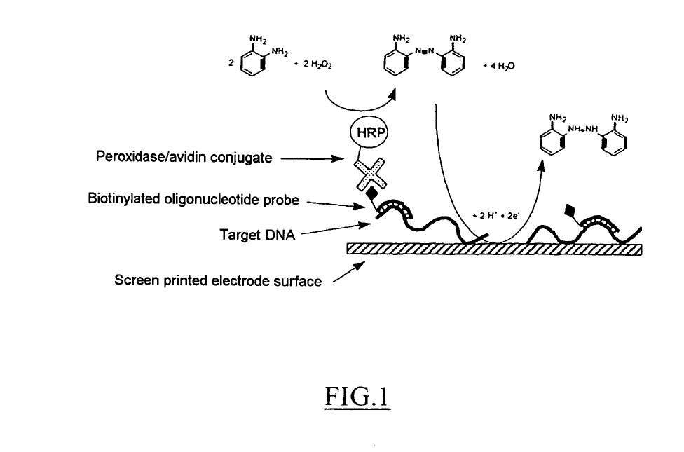

FIG. 2: Voltammograms obtained with the method according to the invention on electrodes coated with (a) the amplified DNA of HCMV (4.10⁹ copies in the solution) and (b) the amplified DNA of the ETS2 gene.

FIG. 3: calibration curves (S/N, signal/noise) for the amplified DNA of HCMV, using several methods. A logarithmic scale is used.
a-c: hybridization on electrode, with calorimetric detection (a) or electrochemical detection (b, c).
d: hybridization and conventional colorimetric detection on microtitration plate
e: quantification of the DNA by ETB fluorescence using agarose electrophoresis.

FIG. 4: Comparative study of the specificity of the method according to the invention, by electrochemical detection (black), or spectrophotometric detection (white) on electrodes, or by the conventional calorimetric method (grey). Amplified fragments of the ETS2 gene, of the EBV virus and of the HCV virus, and a positive control and a negative control of the amplified DNA of HCMV are used. Logarithmic scale.

FIG. 5: Comparative study of the capacity for detection of the amplified DNA of HCMV in human samples (1-10), using the conventional calorimetric method on microplates (white) or the method according to the invention (black). A positive control (+) and two negative controls (−) were included. Logarithmic scale.

### EXAMPLES

### Example 1

### DNA Extraction

The HCMV DNA is extracted from the human embryonic lung fibroblast cell line MRC5 infected with the viral strain AD169, with a commercial DNA extraction kit, according to the manufacturer's recommendations. This technique is known to those skilled in the art, and various manufacturers provide kits for such an extraction. It was observed, in particular, that the Invisorb kit from Invitek or the QiaAmpBlood kit from Qiagen make it possible to obtain good results.

The cells are lysed, adsorbed onto silica and then washed by centrifugation. The DNA is eluted in a suitable buffer and the support is removed. The DNA can then be amplified.

### Example 2

### Amplification of the HCMV DNA by PCR

The primers AC1 (SEQ ID NO 1) and AC2 (SEQ ID NO 2), which amplify a fragment of 406 base pairs of a conserved region located in the HIND III X region of the US genome of the cytomegalovirus (Drouet et al., 1993, J. Virol Methods, 45, 259-76), are used.

The PCR reactions are carried out according to the conventional techniques known to those skilled in the art, on a matrix of DNA as prepared in example 1.35 cycles are performed, having the following characteristics: denaturation at 92° C.-15 sec., hybridization at 55° C.-30 sec, extension at 72° C.-30 sec. The denaturation step is 7 min long for the first cycle, and the extension step of the final cycle is followed by a further period of 2 minutes, in which the temperature is maintained at 72° C.

A negative control, which does not contain any DNA matrix, is included for each series of experiments.

### Example 3

### Quantification of the DNA and Preparation of a Concentration Range for Amplified HCMV DNA

Serial dilutions of the DNA amplified according to Example 2 are prepared, and are studied on agarose gel with ETB, in the presence of a calibrated amount of DNA. It is therefore found that the amplified DNA concentration is 10.5 μmol/ml, which corresponds to 6.3.10¹² copies/ml.

A range (from 6.3.10⁴ to 6.3.10¹² copies/ml) is produced by serial dilutions of the concentrated solution of amplified DNA, in the negative control for the PCR.

### Example 4

### Hybridization on Electrodes and Electrochemical or Calorimetric Detection

The detection of the DNA using the method according to the invention takes place in four steps:
a. immobilization of the target DNA
b. hybridization of the labeled probe
c. incubation of the enzymatic conjugate
d. introduction of the substrate for detection
e. voltammetric or colorimetric detection of the product generated by the enzyme.

2 μl of amplified DNA are denatured in an alkaline medium (0.4 M sodium hydroxide) at ambient temperature, for 10 min.

300 μl of attaching buffer containing 1.5 M of ammonium acetate are then added and the electrodes are immersed. This is left to incubate at 37° C. overnight.

The electrodes are then washed with distilled water, and are incubated for 30 minutes at 37° C. in a hybridization buffer (6×SSC, 0.1% SDS) containing 100 ng/ml of probe AC3 specific for the amplified HCMV sequence (SEQ ID No 3), which has been biotinylated.

A washing cycle, which consists of 5 incubations for 1 minute in 500 l of freshly prepared washing solution (6×SSC, 1% SDS), is then carried out.

Next, the electrodes are incubated for 15 min at ambient temperature in 100 μl of buffer (100 mM Tris HCl, pH 7.5-50 mM NaCl-5 g/l skimmed milk) which contains the streptavidin-peroxidase conjugate (1.6 units/ml), and then a washing cycle, as described previously, is immediately carried out.

The electrode is then immersed in 50 μl of a solution of OPD substrate (40 mM citric acid, 150 mM Na₂HPO₄, 5 mM NaCl, 0.02% H₂O₂, an OPD tablet (Argene-Biosoft) in 10 ml of buffer), and incubated at ambient temperature in the dark for 30 min.

The water-soluble, colored and electroactive reaction product, 2,2′-diaminoazobenzene, is detected by absorption spectrophotometry and by differential pulse voltammetry (DPV), in order to compare the two methods.

For the spectrophotometric reading, the electrodes are withdrawn and the wells are read at 492 nm.

For the reading by DPV, a Pt electrode is used as counterelectrode and an Ag/AgCl electrode is used as reference pseudoelectrode. A μ-Autolab potentiostat (from EcoChemie) is used, connected to an interface on a PC, using the GPSE 3 program (EcoChemie). The DPV is carried out with a 25 mV pulse height, a 5 mV potential step, a 0.05 sec pulse duration, and a 0.5 sec interval between two pulses.

For each series of experiments, 2 negative controls (all the reagents, but no DNA) are included. Thus, the optical values or the current values are divided by the values obtained for this control, and it is considered that the samples are positive when the response/blank ratio is greater than 2.

The following results are obtained:

a. Use of the Method According to the Invention for the Detection of DNA

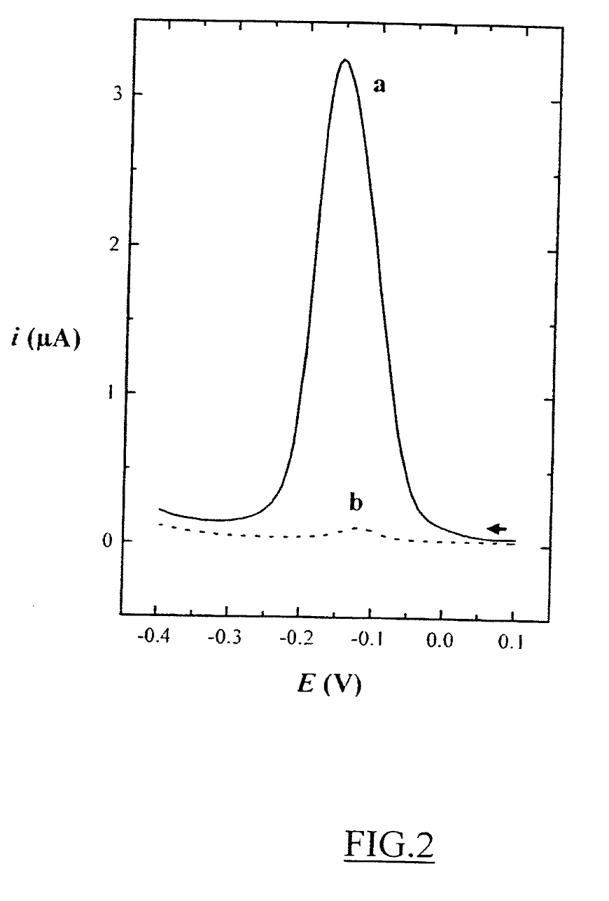

FIG. 2 shows the peaks recorded by DPV, obtained for an electrode brought into contact with the amplified HCMV DNA (FIG. 2a) or for the negative control (FIG. 2b). This figure unambiguously shows that the method according to the invention makes it possible to detect DNA present in solution, by DPV.

b. Specificity and Reproducibility of the Method According to the Invention

In order to measure the specificity and the reproducibility of the method according to the invention, the peaks obtained with 30 electrodes covered either with amplified HCMV DNA or with amplified DNA of a human ETS2 gene were compared. Since the probe AC3 is specific for HCMV, it should not bind to the DNA of the ETS2 gene. There will not therefore be any enzymatic reaction and, consequently, no current peak should be observed for the ETS2 gene.

The following current values are obtained:

| | | |
|---|---|---|
| HCMV: | 3300 ± 100 nA | (3% standard deviation) |
| ETS2: | 61 ± 12 nA | (19% standard deviation) |

This demonstrates that the method is reproducible and that, in the specific hybridization buffer used, the probe does not bind passively to the electrodes, but binds to the complementary sequences already adsorbed onto the electrodes.

c. Detection Limits of the Method According to the Invention, Comparison with the Calorimetric Method

The concentration of amplified HCMV DNA is varied within the range of 0.1 to 10⁶ attomol (6.3.10⁴ to 6.3.10¹¹ copies/ml). The properties of the product generated by the enzymatic reaction are used to compare the voltametric and colorimetric methods.

FIGS. 3a and 3b show the curves obtained with the calorimetric and electrochemical methods respectively.

A comparison with other methods is also carried out:, colorimetry according to the commercial kit Hybridowell™ (ArgeneBiosoft) (3d), or fluorescence densitometry on agarose gel (3e).

It is observed that the calibration curve (3b) for the amplified HCMV DNA is linear, in the 50-2000 attomol range (3.10⁷ amplified DNA molecules), which allows detection of approximately 10 times fewer DNA molecules than by the colorimetric method under the same conditions (3a). The sensitivity of the method is in any case clearly greater than the agarose gel fluorescence (FIG. 3e, detection limit of 14 femtomol), and is equivalent to the colorimetric systems on microtitration plates (FIG. 3d).

In order to increase the sensitivity of the method according to the invention, the volume of OPD substrate introduced for the detection was decreased (10 μl instead of 50 μl). The curve obtained is indicated on FIG. 3c, and it is observed that the detection limit is then taken down to 0.6 attomol (3.6.10⁵ amplified DNA molecules). This is 83 times more sensitive than the calorimetric technique on microtitration plates.

However, in the following experiments, the volume of 50 μl is maintained for the substrate solution.

d. Selectivity and Specificity of the Method According to the Invention

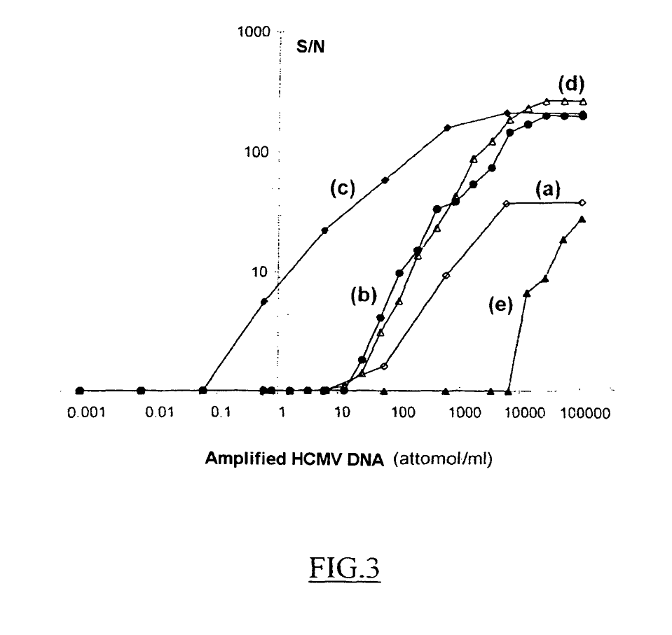

Various DNAs were used to judge the specificity of the method according to the invention, the results being given in FIG. 4. A comparison with the two other calorimetric methods, that described in the present invention and the method on microtitration plates, was carried out. The DNAs used are the amplified DNA of HCMV, the DNA of the human ETS2 gene, and the viral DNAs of the Epstein-Barr virus (EBV) or of the hepatitis C virus (HCV).

It is observed that the method allows specific and selective detection of the HCMV DNA, when the probe specific for this DNA is used, which confirms that this probe does not adsorb passively onto the electrodes during the hybridization step.

e. Characterization of Clinical Samples

The method according to the invention is applied to 10 samples of human serum (4 negatives, 6 positives, as determined previously by quantitative PCR and which contain from 2 to 99 copies/μl of HCMV DNA before amplification) on which the amplification as described in Example 2 was carried out.

The results obtained with the method according to the invention are compared with those obtained using the conventional colorimetric method in microtitration plates.

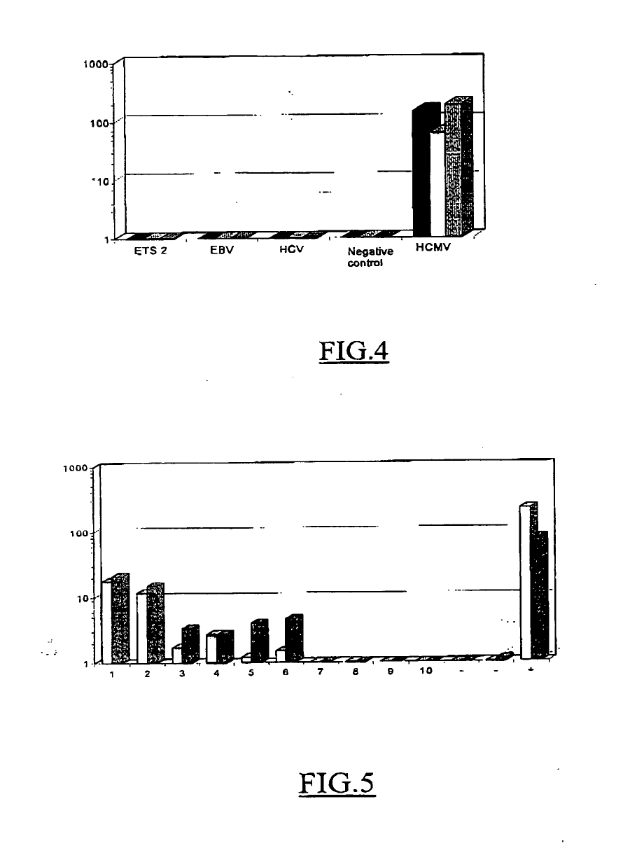

FIG. 5 shows that the method according to the invention does not give any false positives or false negatives, the results obtained for all the samples examined being in accordance with that which was expected.

It is observed, moreover, that the method according to the invention is at least as sensitive as the conventional calorimetric method and that, when the initial number of copies is low, the method according to the invention makes it possible to obtain a better signal/noise ratio (samples 5 and 6).

1. A method for detecting and/or assaying nucleic acids in a sample, comprising the following steps:
a. attaching a nucleic acid to electrodes,
b. specifically hybridizing a nucleic acid complementary to the attached nucleic acid, said complementary nucleic acid containing a recognition agent,
c. adding an agent complementary to the recognition agent of (b), said complementary agent being coupled to an enzyme,
d. adding a substrate for said enzyme such that the action of said enzyme on said substrate leads to the formation of an electroactive compound which can be detected by measuring the variation in faradic current after application of a potential to the electrode.

2. The method as claimed in claim 1, characterized in that it comprises a cascade of enzymatic reactions in step d., before formation of the electroactive compound.

3. The method as claimed in either one of claims 1 and 2, characterized in that the single-stranded nucleic acid attached to the electrodes is the target nucleic acid.

4. The method as claimed in claim 3, characterized in that it also comprises an amplification step before the step of attachment of the nucleic acid to the electrodes.

5. The method as claimed in any one of claims 1 to 4, characterized in that the electrodes are screen printed electrodes.

6. The method as claimed in claim 5, characterized in that the ink for the screen printing contains a mixture of carbon and styrene derivatives.

7. The method as claimed in any one of claims 1 to 6, characterized in that the enzyme which allows the formation of the electroactive compound is an oxidase.

8. The method as claimed in claim 7, characterized in that the oxidase is a peroxidase.

9. The method as claimed in claim 8, characterized in that the peroxidase is horseradish peroxidase.

10. The method as claimed in either one of claims 8 and 9, characterized in that the substrate for the peroxidase is ortho-phenylenediamine, OPD.

11. A kit of reagents, for carrying out the method as claimed in any one of claims 1 to 10, characterized in that it contains:
a. an attaching buffer for attaching the nucleic acid to the electrodes,
b. a hybridization buffer for the specific hybridization of the complementary nucleic acid to the nucleic acid attached to the electrodes,
c. a recognition agent in order to label the complementary nucleic acid,
d. an agent complementary to the recognition agent (d), coupled to an enzyme, and
e. a substrate for said enzyme, leading to the formation of an electroactive compound which allows the variation in faradic current of the solution to be measured.

12. A method for detecting viruses in a biological sample, characterized in that it comprises the following steps:
a. specific amplification of the DNA of the biological sample, preferably by PCR using primers specific for the virus being sought,
b. isolation and analysis of the DNA obtained after amplification using a method as claimed in any one of claims 1 to 10 or using a kit as claimed in claim 11.

13. A method for detecting several different organisms in a sample, characterized in that it comprises the following steps:
a. optional amplification and attachment of the target nucleic acids to the electrodes,
b. hybridization with the nucleic acids complementary to the targets, each one of them being linked to a different recognition agent,
c. addition of the agents complementary to the recognition agents, coupled to different labels,
d. addition of the various substrates for the enzymes in order to generate the various electroactive compounds,
e. measurement of the faradic current corresponding to the various electroactive compounds, at the surface of the electrode, by applying the potential specific for each compound.
